(19) United States
(12) Patent Application Publication
(10) Pub. No.: US 2008/0213367 A1
(43) Pub. Date: Sep. 4, 2008

(54) WATER SOLUBLE CONCENTRIC MULTI-WALL CARBON NANO TUBES

(75) Inventors: Sabyasachi Sarkar, Jamtara (IN); Abdul Allam, Raleigh, NC (US)

Correspondence Address: PASSE' INTELLECTUAL PROPERTY, LLC, 1717 BRASSFIELD RD., RALEIGH, NC, 27614, US

(73) Assignee: Cromoz Inc., Raleigh, NC (US)

(21) Appl. No.: 11/680,888

(22) Filed: Mar. 1, 2007

(51) Int. Cl. A61K 9/00; C07C 51/00

(52) U.S. Cl. 424/484; 562/400; 977/742

(57) ABSTRACT

The present invention relates to water soluble carbon nano tubes (WSNT) which have been made function by functionalizing with carboxylic acid groups. The carbon nano tubes are isolated from hydrocarbon wax soot and then functionalized by oxidation treatment with an oxidizing agent. The WSNT can contain a pharmaceutical composition for release by a biological degradation agent of the WSNT for example E coli.

### BACKGROUND OF THE INVENTION

1. Field of the Invention

The present invention relates to a water-soluble concentric multi-wall carbon nano tube, a method of making them and to their use. More specifically the invention relates to concentric multiwall carbon nano tubes that have been oxidized to add carboxylic acid functionalized groups and to the use of these nano tubes as a means of delivering a pharmaceutical composition.

2. Description of the Related Art

Carbon nano tubes (CNT) are an allotrope of carbon wherein carbon atoms are connected to form a hexagonal honeycomb structure in the shape of a tube or cylinder usually with one end closed. Each CNT is on the order of a few nanometers in diameter or about 50,000 times smaller than a human hair. Given their diameter they can be extraordinarily long, up to several millimeters in length. Most CNT are composed entirely of Sp² bonds similar to graphite. This bonding structure is stronger than the bonds found in diamonds and provides CNT with unusual strength.

There are two main types of CNT known, the single-walled tubes and multi-walled tubes. Multi-wall nano tubes (MWNT) consist of multiple layers of graphite rolled in on themselves. There are two models which normally describe the structures of the MWNT. In the Russian Doll model, sheets of graphite are arranged in concentric cylinders, that is a single walled cylinder within a single walled cylinder. In the Parchment model a single sheet of graphite is rolled around itself resembling a rolled up piece of parchment. While the MWNT has a morphology and properties similar to the single walled nano tubes, the MWNT have significantly increased chemical resistance even when compared to single walled nano tubes. Because of their chemical resistance, functionalization of CNT has been largely ignored. Some attempts have been made though, for example, in U.S. Pat. No. 7,125,533 there is described the fictionalization of CNT single walled nano tubes utilizing peroxides. This method though does not produce any water soluble CNT and does not appear to work with MWNT. In addition, the fictionalization only appears to occur at the end of the tubes and at positions of weakness or structural non-integrity.

Because of CNT's unique structural properties, they lend themselves to a number of applications. The can be used in electrical systems where their high electrical current density of more than 1,000 times silver and copper is extraordinarily useful. CNT are known for their applicability in electron emitters, vacuum fluorescent displays, luminous sources, field emission displays, nano-wires etc. Because of the structural rigidity and chemical stability of CNT they can act as either a conductor or a semiconductor including uses in transistors, and energy reservoirs.

Several applications of CNT's have also appeared in the bioengineering field. Applications including biosensors, protein detection, DNA detection, chemical immobilization and the like all have all been proposed.

However, because the CNT is insoluble in all organic solvents including water, the utility of CNT has some serious limitations to their application. Accordingly, there have been attempts at preparing water soluble nano tubes. One way of water solubilising a CNT is to wrap the otherwise insoluble CNT in a water soluble layer. In U.S. patent application Ser. 2006/0003401 there is described a CNT that has been wrapped with a so called “self-assembly material”. However, the CNT itself is not water soluble and once wrapped the CNT is isolated from its environment.

There are various methods current of producing a CNT both MWNT and SWNT. In the production of MWNT conventional methods such as the carbon arc method, laser ablation, and chemical vapor deposition are used to make a graphite structure nano tube usually starting with graphite as the starting material. Carbon nano tubes are also produced by flames, burning methane, ethylene and benzene and have been found in soot from both indoor and outdoor air. However these varieties are normally considered to be relatively useless because of their irregular size and low quantity.

Soot is the dark powdery deposit of unburned fuel residues, usually composed mainly of amorphous carbon from the combustion of various carbon-rich organic fuels that lack sufficient oxygen. To date there does not appear to be any significant use of carbon nano tubes that are produced by soot.

The art while suggesting various strategies used to solubilize MWNT does not teach or suggest functionalized MWNT that could be water soluble nor suggest any uses for such MWNT. It would be useful if there were a method for producing such water soluble MWNT.

### SUMMARY OF THE INVENTION

It has been discovered that a certain type of nano tube can be made water soluble by functionalizing the surface of the nano tube. Specifically, MWNT produced from hydrocarbon wax soot can be oxidized to produce side wall carboxylic acid groups on the outer surface thus making the MWNT produced water soluble. It has further been discovered that these water soluble nano tubes (WSNT) can contain a pharmaceutical composition and be used as a drug delivery system.

The present invention relates to novel methods of making water soluble MWNT and to novel MWNT and their uses. In one embodiment, the manufacture of the water soluble MWNT involves a method of making a water soluble concentric multi-wall carbon nano tube comprising:
a. heating a hydrocarbon wax to a temperature of between about 500 degree C. and 800 degree C. under conditions sufficient to produce carbon soot containing carbon nano tubes;
b. purifying the soot to isolate the nano tubes;
c. oxidativly treating the nano tubes to remove amorphous carbon and produce hydrophilic carboxylic acid groups on the sidewall surface of the nano tubes.

The novel WSNT of the present invention has several utilities but in one embodiment the WSNT comprises soot produced concentric multi-wall carbon nano tubes that have been made water soluble by the addition of functionalized carboxylic acid groups to the sidewall surface.

In yet another embodiment of the invention, there is soot produced concentric multi-walled carbon nano tubes that have been made water soluble by the addition of carboxylic acid groups to the sidewall surface and further comprising a pharmaceutical composition within the WSNT.

In yet another embodiment of the invention, there is described a method of delivering a pharmaceutical composition to a patient in need thereof comprising administering to the patient the pharmaceutical composition contain in a soot produced concentric multi-walled carbon nano tubes that have been made water soluble by the addition of carboxylic acid groups to the sidewall surface.

These and other objects of the present invention will be clear when taken in view of the detailed specification and disclosure in conjunction with the appended figures.

### BRIEF DESCRIPTION OF THE DRAWINGS

FIG. 1 is an X-ray diffraction patter of the WSNT of the invention.

FIG. 2 is a TEM photograph showing the WSNT of the invention

FIG. 3 is a HRTEM photograph showing a further Hi-resolution image of the WSNT of the invention.

FIG. 4 is a TEM of E-coli in the presence of the WSNT of the invention.

FIG. 5 is a TEM of E-coli opening up the WSNT of the present invention to deliver the drug carried by the WSNT of the invention.

### DETAILED DESCRIPTION OF THE INVENTION

The general description of the invention and how to use and make the WSNT of the invention are stated in the Brief Summary above. This detailed description defines the meaning of the terms used herein and specifically describes embodiments in order for those skilled in the art to practice the invention. The above interests in providing WSNT and using them to administer pharmaceutical compositions and how to administer treatment are explained and met as can be seen readily from the disclosure which follows and thus met by the present invention.

As used herein the term “hydrocarbon wax” refers to those hydrocarbons having properties similar to beeswax namely malleable at normal ambient temperature, a melting point above about 45 degrees C., a low viscosity when melted, insoluble in water and hydrophobic. Waxes can be natural origin or artificial. Chemically a way may be an ester of ethylene glycol and two fatty acids. Several different categories of hydrocarbon waxes are known for example animal waxes, insect waxes, mineral waxes, petroleum waxes and synthetic waxes. In one embodiment the wax can be selected but not limited to beeswax, carnauba wax, castor wax, jojoba wax, rice bran wax, ceresin waxes, ozocerite wax, paraffin wax, microcrystalline wax, polyethylene wax and the like.

As used herein the phrase “conditions sufficient to produce carbon soot” refers to the burning of the hydrocarbon wax under conditions of oxygen supply sufficient to produce soot. Soot in general results from the incomplete combustion of the hydrocarbon in the wax. Typically this means that the flame is deprived of sufficient oxygen to combust entirely. It also is useful that convection type conditions exist during the combustion effort such as would exist with an open flame. For example, the burning of a bees wax candle creates a convection or uplifting draft which carries the soot away from the flame and aids in the prevention of complete combustion. Typically the flame used to produce soot is at a temperature of between about 500 and 800 degrees C. usually as an open flame however other sources of heat that produce soot are well known. The result of this production is the production of amorphous carbon and of multiwalled concentric carbon nano tubes of the type consistent with the Russian Doll model.

As used herein the phrase “purifying the soot” refers to a solvent washing of the soot to separate the amorphous carbon that is not nano tubes from the amorphous carbon that has formed an amorphous carbon nano tube (and not a graphite nano tube). Suitable solvents include separating the amorphous carbon from the MWNT by either differences in density, weight or the like of the two products.

As used herein “oxidativly treating” refers to treatment of the isolated nano tubes with an oxidizing agent such that the defective portion of the nano tube being more reactive is exposed to an oxidizing agent to produce carboxylic acid groups attached at that point. The point of attachment can be any portion on the outer surface, side wall, ends or end wall or the like. This includes the kinks bends and open ends but also includes the rest of the outer surface of the nano tube normally resistant to fictionalization in other graphite nano tubes. Oxidizing agents suitable for use in the invention include but are not limited to nitric acid, per acids, ammonium cerium nitrate, nitrogen dioxide, chlorine oxide. Oxidizing agents can be mixed together as well so in one embodiment nitric acid could be combined with sulfuric acid, sodium meta bisulfate or a per acid. The concentration and time of exposure of the oxidizing agent to the nano tube depends on the temperature, the oxidizing agent selected, the time of exposure, the amount of functionilization desired and the like but is well within the skill in the art in view of the disclosure herein.

As used herein the term “carboxylic acid group” refers in general to the chemistry of oxidizing a wax hydrocarbon nano tube as follows: On prolonged (48-50 hours) refluxing with dilute nitric acid (2.6M) resulted in carboxylation of the surface of the CNT. On evaporation and extraction of the soluble part by treating the mass under soxhlet with water, results in the separation of the water-soluble fraction, which can be finally evaporated to dryness and washed with ethanol to free it from some, oxidized organic acids. The nitration on carbon centers results in nitro group. This nitro group changes to isonitro (isomerization) followed by its hydrolysis to hydroxo, which on further oxidation results in a carboxylic acid group. The yield of the soluble part varies with the time of oxidation. This ranges from 20 to 40 percent of the starting CNT's. The oxidation can be carried out by a mixture of nitric acid/sulfuric acid or by sulfuric acid and potassium hydrogen sulfate or by persulfate and sulfuric acid or by hydrogen peroxide and sulfuric acid mixture or other per acid salts like perborate or perhalate and acid. Besides acids, nitrogen dioxide chlorine dioxide can be used as oxidant to introduce labile functional groups which under hydrolysis and finally under acid treatment led to form relatively more stable carboxylate acid groups.

The present MWNT of the invention can be made according to the following schedule. A hydrocarbon wax is burned in a flame or other appropriate heat source for example a thread burning in wax as would be the case with a candle. Such a flame burns at about 400 to 800 degrees C. which here can be varied by adjusting the flame source, e.g. by increasing the wick or utilizing a hotter flame source. As the hydrocarbon wax burns it creates an updraft or convection pulling soot and oxygen away from the flame. This creates lack of oxygen at the flame and further creates soot and production of soot nano tubes. The burning of the hydrocarbon could also be done in a closed environment that has a measured amount of oxygen such that soot production is controlled to the degree desired for MWNT production. The soot is then collected by a convenient method for example by placing a surface above the rising soot which would then collect the soot. The soot is then washed with appropriate solvent and MWNT of about 30 to 60 nm and a length of about 0.5 to about 10 microns are isolated. The MWNT can be isolated by any convenient method so for example they could be separated by density difference, weight difference or the like. Clearly one advantage of the present invention is that these nano tubes of the invention are amenable to production on an industrial scale due to the ease of production. The isolated MWNT could be precipitated out of solution or used in this state to add functionalized carboxylic groups as described elsewhere herein.

The isolated MWNT are then treated with appropriate oxidizing agent and the resultant MWNT are functionalized with carboxylic acid groups as described above. The resultant WSNT are highly soluble in water and have a pore size of about 8 nm to about 2 nm. Other molecules may be trapped within the WSNT and the WSNT may be used to deliver the molecule to a particular site in a mammal in need of delivery of the molecule. The normal bends and kinks present in the WSNT make these bend and kink points vulnerable for subsequent opening of the WSNT and release of the contents. In addition the carboxylic acid groups and the process to add them create other weaknesses in the WSNT which can be points of attack to open the WSNT releasing its content under proper circumstances. This could occur where, in one embodiment, the WSNT was filled with a pharmaceutical composition and introduced into an organism. The normal biological processes, for example, degradive acids or enzymes or for example flora of the intestine such as E. coli could attack the weak points of the WSNT, opening the WSNT, and then releasing the pharmaceutical composition for introduction to the organism at the site the WSNT are delivered. By making the WSNT under different temperatures and varying other conditions including starting waxes and the like, the pore size and number and placement of bends and kinks can be adjusted thus selectively adjusting the period of time for release of the pharmaceutical composition within the WSNT to the organism or to a particular site in the organism.

As mentioned above the WSNT could include inside of the WSNT a pharmaceutical composition for delivery to a animal in need of the pharmaceutical composition. A pharmaceutical composition, which comprises a pharmaceutical compound and a pharmaceutically acceptable carrier, diluent or excipient could also be included. A liquid formulation of a pharmaceutical composition may be a buffered, isotonic, aqueous solution. Examples of suitable diluents are normal isotonic saline solution, standard 5% dextrose in water or buffered sodium or ammonium acetate solution. Such formulation is especially suitable for oral administration. It may be desirable to add excipients such as polyvinylpyrrolidone, gelatin, hydroxy cellulose, acacia, polyethylene glycol, mannitol, sodium chloride or sodium citrate. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit.

The amount of a compound of the invention (hereinafter referred to as the active ingredient) required for therapeutic or prophylactic effect upon administration will, of course, vary with the compound chosen, the nature and severity of the condition being treated and the mammal undergoing treatment, and is ultimately at the discretion of the physician and within the skill of those in the art armed with the disclosure of the invention herein.

### EXAMPLES

A bee's wax candle was lit and allowed to burn via its central cotton wick under normal open air conditions. Carbon soot is collected on a glass reflector plate is fixed over the candle and repositioned as needed during the burning process. The temperature during this burning process was recorded and was about 600 degrees Centigrade during the entire process. The collected soot was then washed repeatedly with toluene, acetonitrile and ethanol by soxhlet method to free the soot from any unburnt amorphous hydrocarbon or fullerenes and then dried. The MWNT were then treated with a solution of dilute (2.6 M) nitric acid under reflux for 24 hours and then the entire mass was dried. The treated soot was then treated with water first followed by ethanol under soxhlet to free it from nitric acid ad any readily soluble organic derivatives that may be present. The remaining soot was allowed to dry. The resulting soot consists mainly of WSNT functionalized by carboxylic acid groups on the surface of the now WSNT. The average yield is one gram of WSNT from burning 50 grams of bee's wax.

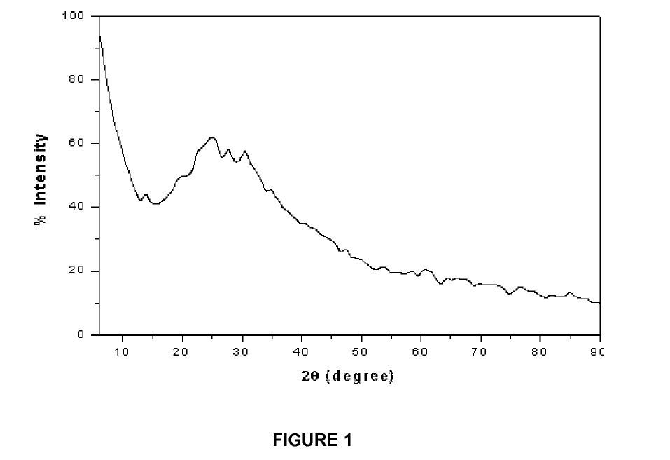

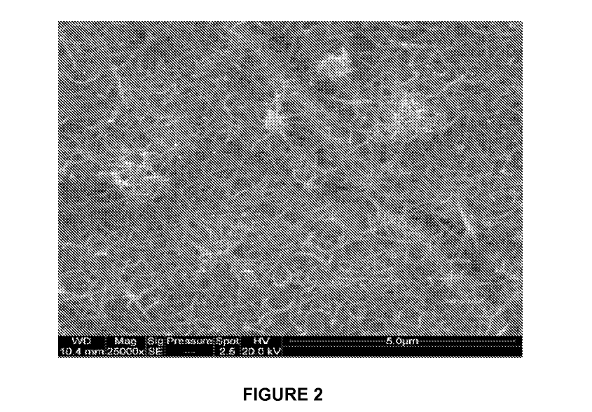

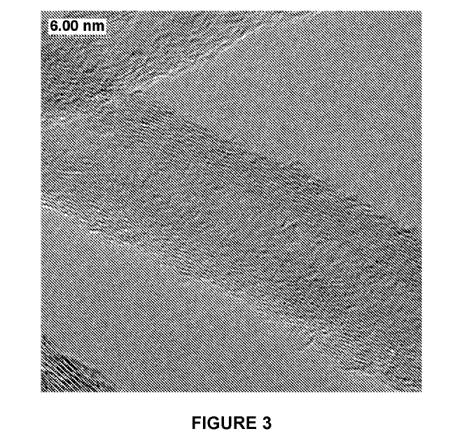

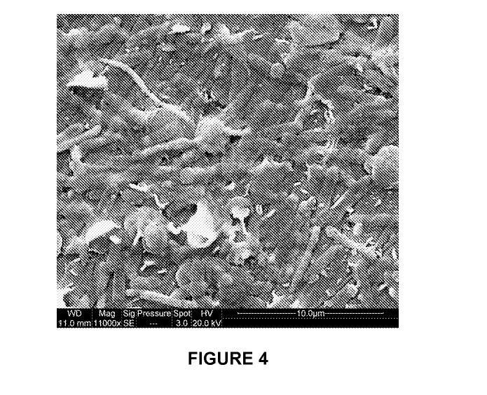

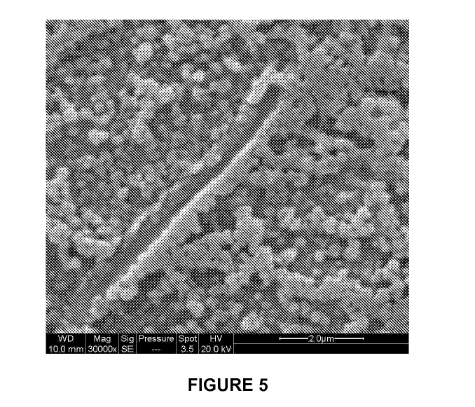

These WSNT can then, in solution, be filled with a pharmaceutical composition. The resultant WSNT are X-rayed and the diffraction pattern is seen in FIG. 1. The WSNT are then photographed under Scanning Electron Micrograph (SEM) and shown in FIG. 2. A High Resolution Transmission Electron Micrograph is shown in FIG. 3. The release of the enclosed pharmaceutical composition is then accomplished by treatment with E coli. In FIG. 4 there is a SEM photograph of the effect of first generation E coli on the WSNT. By the time the E coli are on the third generation, the effect of the E coli on the WSNT is evident and the tubes have opened to release their contents as seen in the final SEM photograph FIG. 5.

Examples and the explanations herein are intended to be illustrative of the present invention and not to be construed as limiting in any manner. The invention is defined by the following claims with equivalents to each claim and part of the claim intended to be included therein.

What is claimed is:

1. A method of making a water soluble concentric multi-wall carbon nano tube comprising:
a) heating a hydrocarbon wax to a temperature of between about 500 degree C. and 800 degree C. under conditions sufficient to produce carbon soot containing carbon nano tubes;
b) purifying the soot to isolate the nano tubes;
c) oxidatively treating the nano tubes to remove amorphous carbon and produce hydrophilic carboxylic acid groups on the sidewall surface of the nano tubes.

2. A method according to claim 1 wherein the nano tubes are oxidativly treated with one or more oxidizing agent selected from the group consisting of:
a) nitric acid;
b) a per acid;
c) ammonium ceric nitrate.

3. A method according to claim 1 wherein the hydrocarbon wax is selected from the group comprising:
a) an insect wax;
b) an animal wax;
c) a mineral wax;
d) a synthetic wax;
e) a vegetable wax; and
f) a petroleum wax.

4. A method according to claim 3 wherein the petroleum wax is paraffin.

5. A method according to claim 3 wherein the insect wax is bees wax.

6. A method according to claim 1 wherein the method further comprises incorporating a pharmaceutical composition within the nano tubes.

7. A method according to claim 1 wherein the soot is purified by solvent washing.

8. A method according to claim 2 wherein the oxidative agent is nitric acid.

9. A method according to claim 8 wherein the nitric acid is mixed with one or composition selected from the group comprising:
a) sulfuric acid;
b) sodium meta bisulfate; and
c) a per acid like perboric acid, peroxy sulfuric acid, hydrogen peroxide and its other derivatives containing peroxo group.

10. A method according to claim 2 wherein the oxidative agent is a per acid.

11. A method according to claim 10 wherein the per acid is combined with nitrogen dioxide.

12. Soot produced concentric multi-wall carbon nano tubes that have been made water soluble by the addition of carboxylic acid groups to the sidewall surface.

13. Carbon nano tubes according to claim 12 with a diameter of from about 30 to 60 nm.

14. Carbon nano tubes according to claim 12 which further comprises a selected pharmaceutical within the nano tubes.

15. Carbon nano tubes according to claim 14 wherein the selected pharmaceutical is in a liquid form.

16. Carbon nano tubes according to claim 14 wherein the selected pharmaceutical is selected from the group comprising antibiotics, and photodynamic therapeutic reagents.

17. A method of delivering a pharmaceutical composition to a patient in need thereof comprising administering to the patient the pharmaceutical composition contained in a soot produced concentric multi-walled carbon nano tubes that has been made water soluble by the addition of carboxylic acid groups to the sidewall surface of the nano tube.
